(19) United States
(12) Patent Application Publication
(10) Pub. No.: US 2020/0069630 A1
(43) Pub. Date: Mar. 5, 2020

(54) COMPOSITIONS FOR SEXUALLY TRANSMITTED DISEASES

(71) Applicant: Hennipen Life Sciences, LLC, Plymouth, MN (US)

(72) Inventors: Marnie L. Peterson, Jackson, WY (US); Patrick M. Schlievert, Iowa City, IA (US)

(21) Appl. No.: 16/316,475

(22) PCT Filed: Jul. 10, 2017
(86) PCT No.: PCT/US17/41354

§ 371 Date: Jan. 9, 2019

Related U.S. Application Data
(60) Provisional application No. 62/375,082, filed on Aug. 15, 2016.
(60) Provisional application No. 62/360,561, filed on Jul. 11, 2016.

(51) Int. Cl. A61K 31/23; A61K 47/10; A61K 47/12; A61K 47/18; A61K 47/44; A61K 9/00

(52) U.S. Cl. CPC A61K 31/23; A61K 47/10; A61K 9/0014; A61K 47/183; A61K 47/44; A61K 47/12

(57) ABSTRACT

Disclosed are compositions and methods for treatment microbial infections. The compositions comprise glycerol monolaurate or a derivative thereof, and can be administered topically, for example, to treat gonorrhea and Zika.

### CROSS-REFERENCE TO RELATED APPLICATIONS

This application claims priority to International Application No. PCT/US2017/041354, filed Jul. 10, 2017, entitled “COMPOSITIONS FOR SEXUALLY TRANSMITTED DISEASES,” which in turn claims the benefit of U.S. Provisional Patent Application Ser. No. 62/360,561, filed Jul. 11, 2016 and U.S. Provisional Patent Application Ser. No. 62/375,082, filed Aug. 15, 2016, the entireties of each application are incorporated herein by reference.

### BACKGROUND

Sexually transmitted diseases (STDs) can be caused by bacteria, viruses, or parasites. Examples include gonorrhea, genital herpes, human papillomavirus infection, HIV/AIDS, chlamydia, and syphilis. Gonorrhea is a sexually transmitted infection caused by the bacterium Neisseria gonorrhoeae. Untreated, gonorrhea can cause serious and sometimes permanent health problems in men, women and babies. For example, in men, gonorrhea may be complicated by epididymitis, and in rare cases, infertility. In women, gonorrhea is a major cause of pelvic inflammatory disease, chronic pelvic pain, ectopic pregnancy, stillbirths, and tubal infertility. In babies of infected mothers, gonorrhea can cause neonatal eye infections that can lead to blindness. Gonorrhea can also spread to the blood and cause disseminated gonococcal infection, usually characterized by dermatitis-arthritis syndrome, tenosynovitis, and more rarely, bacteremia, meningitis or endocarditis, and can be life-threatening. Gonorrhea infections can also facilitate transmission of HIV.

Recently, Zika virus infections in humans have increased. Zika has been shown to be sexually transmitted. The Zika virus is an arthropod-borne virus (arbovirus) in the genus Flavivirus, an enveloped virus. The virus was initially isolated from nonhuman primates and from mosquitoes. Zika is primarily spread by the female Aedes aegypti mosquito.

### SUMMARY

Disclosed is a composition and method for treating microbial infections. In one aspect of the invention is disclosed a method of treatment or prophylaxis of a sexually transmitted disease in a subject, wherein the sexually transmitted disease is caused a by a bacterium, a fungus, or a protozoan, the method comprising administering to the subject a composition comprising an effective amount of glycerol monolaurate and derivatives thereof and one or more pharmaceutically acceptable excipients.

In one aspect of the invention is disclosed a use of a composition in treating sexually transmitted disease comprising an effective amount of glycerol monolaurate and derivatives thereof and one or more pharmaceutically acceptable excipients.

### DETAILED DESCRIPTION

Disclosed are compositions and methods of treatment of microbial infections with the compositions containing glycerol monolaurate (GML), which may be administered, e.g., by topical administration. The compositions and methods provided herein, in one embodiment, are used for treating infections topically, for example, by facilitating delivery of effective amounts of GML or a derivative thereof to a skin or mucosal surface of a subject, e.g., a human. It is expected that the compositions may result in greater patient compliance for topical self-administration due to the less irritating nature of the composition, relative to previously employed topical formulations of anti-microbial and anti-viral compounds.

As used herein, the term “antimicrobial” means effective in preventing, inhibiting, or arresting the growth or pathogenic effects of a microorganism. “Microorganism” is used herein to mean any bacteria, virus, fungus, or protozoa.

“Anti-bacterial” or “anti-fungal, or “anti-viral,” or “anti-protozoan” as used herein, refers to inhibition or arrest of the growth of a bacterium or fungus or virus or protozoan, a reduction in the severity of or likelihood of developing a bacterial or fungal or viral or protozoan disease, inducing death of the bacterium or fungus or virus or protozoan or reduction or inhibition of the pathogenic effects of the respective bacterium, fungus virus or protozoan. “Bactericidal” is used interchangeably with “anti-bacterial.”

The term “effective amount,” as used herein, refers to an amount that is sufficient to affect a beneficial or desired antimicrobial activity, including, without limitation, killing the microorganism or inhibiting microbial infection, growth or toxicity.

The terms “treat,” “treatment,” and “treating” refer to an approach for obtaining beneficial or desired results, for example, clinical results. For the purposes of this application, beneficial or desired results may include inhibiting or suppressing the growth of a microorganism or killing a microorganism; inhibiting one or more processes through which a microorganism infects a cell or subject; inhibiting or ameliorating the disease or condition caused by a microbial infection; or a combination thereof. The terms “treat,” “treatment,” or “treating” also refer to prophylaxis of infection.

“Prophylaxis,” as used herein, can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein, the term “subject” includes humans and other animals.

“Topical,” as used herein, refers to the application of the composition to any skin or mucosal surface.

“Skin surface” refers to the protective outer covering of the body of a vertebrate, generally containing a layer of epidermal cells and a layer of dermal cells. A “mucosal surface,” as used herein, refers to a tissue lining of an organ or body cavity that secretes mucous, including but not limited to oral, vaginal, rectal, gastrointestinal, and nasal surfaces.

The term “pharmaceutically acceptable topical carrier,” as used herein, refers to a material, diluent, or vehicle that can be applied to skin or mucosal surfaces without undue toxicity, irritation, or allergic reaction.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

As used herein, the term “swab” refers to a material suitable for applying a liquid, gel, wax, cream, or lotion to a skin or mucosal surface, or the act of applying a liquid, gel, wax, cream, or lotion to the skin or mucosal surface, or the act of collecting a liquid, gel, wax, cream, lotion, or fluid from the skin or mucosal surface.

As used herein, the term “vegetable oil” means a substance extracted from a plant or seed that exists in liquid form at room temperature.

The term “accelerant,” as used herein, refers to a compound, substance, liquid, powder, or mixture that, when added to the composition, has the effect of enhancing or contributing to the antimicrobial properties of the composition.

As used herein, the term “cellulose derivative” refers to any a cellulose-based compound and may include, for example, hydroxyethyl cellulose, hydroxypropyl cellulose, methylcellulose, ethylcellulose, hydroxypropyl methyl cellulose, or cellulose acetate.

In one embodiment, the present invention provides a topical composition comprising glycerol monolaurate (GML) or a derivative thereof. In a further embodiment, the composition comprises a vegetable oil or a non-aqueous gel, or a combination thereof. The non-aqueous gel, in one embodiment, comprises a cellulose derivative. The topical composition provided herein, in one embodiment, comprises a pharmaceutically acceptable topical carrier.

In one embodiment, the composition provided herein comprises the monoglyceride GML. GML is a fatty acid ester of glycerol, derivative of lauric acid, with the chemical formula C₁₅H₃₀O₄. GML is also known in the art as glyceryl laurate or monolaurin. GML is found naturally in breast milk and some plants, and is used as a food and cosmetic additive. GML and other glycerides are listed in the Generally Recognized as Safe Substances database by the US Food and Drug Administration. GML and related compounds have been previously disclosed in U.S. patent application Ser. No. 10/579,108 (filed Nov. 10, 2004), and Ser. No. 13/866,722 (filed Apr. 19, 2013), and U.S. Pat. No. 8,796,332 (filed Aug. 2, 2005), the disclosures of each of which are herein incorporated by reference for all purposes.

GML can be synthesized in multiple forms including both R and S optical isomers, as well as forms with lauric acid in the 1/3-position and in the 2-position. The composition provided herein, in one embodiment, comprises the R isomer of GML. In another embodiment, the composition provided herein comprises the S isomer of GML. In yet another embodiment, a racemic mixture of isomers is provided in the composition.

Similarly, the topical composition may comprise GML with lauric acid at the 1/3 position, GML with lauric acid at the 2-position, or a combination thereof R and S isomers of each form, and racemic mixtures thereof, are amenable for use with the present composition.

The chemical structure of GML with lauric acid in the 1/3-position is:

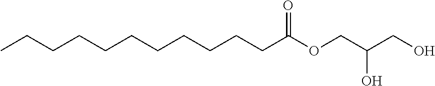 [SMILES: CCCCCCCCCCCC(=O)OCC(O)CO]

### Glycerol Monolaurate (GML) 1/3-Position

The chemical structure of GML with lauric acid in the 2-position is:

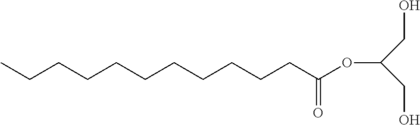 [SMILES: CCCCCCCCCCCC(=O)OC(CO)CO]

### Glycerol Monolaurate (GML) 2-Position

In another embodiment, the topical composition comprises a GML derivative, for example a compound selected from one of Formulae I-VI. Examples of such compounds include, by way of example and without limitation, glycerol monocaprylate, glycerol monocaprate, glycerol monomyristate, glycerol monopalmitate, and dodecyl glycerol.

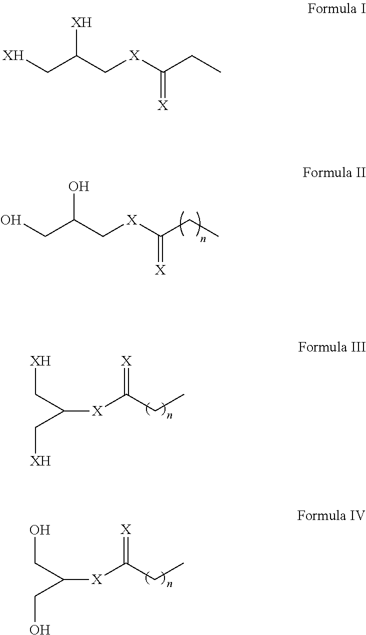

wherein each occurrence of X is independently —O— or —S—; and n is an integer from 5 to 20 (inclusive).

In another embodiment, the topical composition comprises at least one derivative of GML, and the at least one derivative is a compound of either Formula V or Formula VI. Examples of such compounds include, but are not limited to, glycerol dilaurate, glycerol dicaprylate, glycerol dimyristate, glycerol trilaurate, and glycerol tripalmitate.

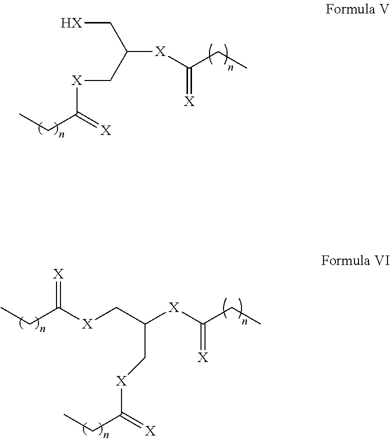

wherein each occurrence of X is independently —O— or —S—; and each occurrence of n is independently an integer from 5 to 20 (inclusive).

In one embodiment, a compound of Formula I, II, III or IV is present in the topical composition of the invention, and at least one —X— is —S—. In one embodiment, one occurrence of —X— is —S— and the remaining occurrences of —X— are —O—.

In one embodiment, a compound of Formula V or VI is present in the topical composition, each occurrence of n is 10, and at least one —X— is —O—.

The topical composition provided herein, in one embodiment, comprises GML and a GML derivative. For example, in one embodiment, the topical composition provided herein comprises GML and a compound of Formula VI. In a further embodiment, each occurrence of n is 10 and at least one —X— is —O—.

In one embodiment, the topical composition comprises GML or a derivative thereof from about 0.001% (w/v) to about 10% (w/v) of the composition. In a further embodiment, GML or a derivative thereof comprises about 0.005% (w/v) to about 5% (w/v) of the composition. In a still further embodiment, GML or a derivative thereof comprises about 0.01% (w/v) to about 1.0% (w/v) of the composition. In yet a further embodiment, GML or a derivative thereof comprises about 0.05% (w/v) to about 0.5% (w/v) of the composition.

In another embodiment, the topical composition comprises GML or derivative thereof at a concentration of about 10 μg/mL, to about 100 mg/mL. In a further embodiment, the topical composition comprises GML or derivative thereof at a concentration of about 50 μg/mL to about 50 mg/mL. In a further embodiment, the topical composition comprises GML or derivative thereof at a concentration of about 100 μg/mL to about 10 mg/mL. In yet a further embodiment, the topical composition comprises GML or a derivative thereof at a concentration of about 500 μg/mL to about 5 mg/mL.

In one embodiment, the topical composition comprises GML or derivative thereof at a concentration of about 10 μg/mL, about 50 μg/mL, about 100 μg/mL, about 500 μg/mL, about 1 mg/mL, about 5 mg/mL, about 10 mg/mL, about 50 mg/mL, or about 100 mg/mL. An effective amount of GML is about 10 μg/mL, about 50 μg/mL, about 100 μg/mL, about 500 μg/mL, about 1 mg/mL, about 5 mg/mL, about 10 mg/mL, about 50 mg/mL, or about 100 mg/mL.

The amount of GML or derivative thereof in the composition can be tailored accordingly to the indication/disease being treated as well as the characteristics of the subject being treated. The amount of GML or derivative in the composition may vary depending on, for example, the nature of the infection or illness; the site of administration; the subject's medical history, subject weight, age, sex, and surface area being treated; and whether the subject is receiving any other medications.

As provided above, one embodiment is directed to a topical composition comprising GML or a derivative thereof. In one embodiment, the topical composition comprises at least one glycol. For example, in one embodiment, the topical composition comprises propylene glycol, polyethylene glycol, or a combination thereof. In one embodiment, the polyethylene glycol has a molecular weight (MW) range from about 300 to about 10,000. In a further embodiment, the polyethylene glycol has a molecular weight of about 300 to about 1,000. In a still further embodiment, the polyethylene glycol has a molecular weight of about 400.

In one embodiment, polyethylene glycol is present in the topical composition. In a further embodiment, the polyethylene glycol has a MW of about 400, about 500 or about 1,000. In one embodiment, the polyethylene glycol is present in the topical composition at a concentration (w/w) of about 15% to about 50%, about 20% to about 40%, or about 25% to about 35%, for example, about 15%, about 20%, about 25%, about 30%, about 35%, about 40%, about 45%, or about 50%. In a further embodiment, both propylene glycol and polyethylene glycol are present in the topical composition. In a further embodiment, propylene glycol is present at a concentration of about 70% to about 80% and polyethylene glycol is present at a concentration of about 20% to about 30%. In even a further embodiment, the polyethylene glycol is polyethylene glycol 400.

In another embodiment, a topical composition comprising GML or a derivative thereof is provided. In a further embodiment, propylene glycol is present in the composition. In yet a further embodiment, propylene glycol is present in the composition at a concentration of about 60% to about 80%, for example, about 60%, about 65%, about 70%, about 71%, about 72%, about 73%, about 74%, about 75%, or about 80%.

In some embodiments, the propylene glycol can help spread to coat various reproductive organs such as the vagina, uterus, fallopian tubes, and urethra. This effective spreading can enhance elimination of bacteria such as bacteria from the genus Neisseria and other microbial organisms such as fungus, virus and protozoa. One of skill in the art would accomplish the effective spreading as shown, for example, in Barnhart K, Pretorius E S, Stolpen A, Malamud D, 2001 Distribution Of Topical Medication In The Human Vagina As Imaged By Magnetic Resonance Imaging, Fertil Steril 76:189-195; Barnhart K T, Stolpen A, Pretorius E S, Malamud D. 2001, Distribution Of A Spermicide Containing Nonoxynol-9 In The Vaginal Canal And The Upper Female Reproductive Tract, Hum Reprod 16:1151-1154; Pretorius E S, Timbers K, Malamud D, Barnhart K. 2002, Magnetic Resonance Imaging To Determine The Distribution Of A Vaginal Gel: Before, During, And After Both Simulated And Real Intercourse, Contraception 66:443-451; and Mauck C K, Katz D, Sandefer E P, Nasution M D, Henderson M, Digenis G A, Su I, Page R, Barnhart K. 2008, Vaginal Distribution Of Replens And K-Y Jelly Using Three Imaging Techniques, Contraception 77:195-204. In some embodiments, the effective spreading can enhance elimination of virus such as from the genus Flavivirus.

In one embodiment, the topical composition comprises at least one cellulose derivative. In a further embodiment, the composition comprises one cellulose derivative or two cellulose derivatives. In one embodiment, the cellulose derivative is hydroxypropyl cellulose. In another embodiment, the cellulose derivative is hydroxyethyl cellulose, carboxymethyl cellulose or hydroxymethyl cellulose. In yet another embodiment, the composition comprises a combination of hydroxyethyl cellulose and hydroxypropyl cellulose. In one embodiment, the cellulose derivative is present at a concentration of about 0.1% (w/w) to about 5.0% (w/w). In a further embodiment, multiple cellulose derivatives are present in the composition at the same concentration. In a further embodiment, two cellulose derivatives are present, and each is present at a concentration of about 1.25% (w/w). Cellulose derivatives include, for example, hydroxyethyl cellulose, hydroxypropyl cellulose, methylcellulose, ethylcellulose, hydroxypropyl methyl cellulose, or cellulose acetate.

In one embodiment, the topical composition provided herein includes GML or a derivative thereof, at least one cellulose derivative, propylene glycol and polyethylene glycol.

In another embodiment, a topical composition comprising GML or a derivative thereof is provided. In a further embodiment, the composition comprises at least one vegetable oil, for example, at least one of the vegetable oils described above (e.g., palm oil, olive oil, corn oil).

Suitable vegetable oils include, without limitation, palm, olive, corn, canola, coconut, soybean, wheat germ, jojoba, sunflower, sesame, peanut, cottonseed, safflower, soybean, rapeseed, almond, beech nut, cashew, hazelnut, macadamia, mongongo nut, pecan, pine nut, pistachio, walnut, grapefruit seed, lemon, orange, bitter gourd, bottle gourd, buffalo gourd, butternut squash seed, egusi seed, pumpkin seed, watermelon seed, acai, black seed, blackcurrant seed, borange seed, evening primrose, flaxseed, eucalyptus, amaranth, apricot, apple seed, argan, avocado, babassu, coriander seed, grape seed, mustard, poppy seed, rice bran, castor, or mixtures thereof. Mixtures can be, by way of example and without limitation, a combination of olive oil and soybean oil, a combination of coconut oil and wheat germ oil, or a combination of jojoba oil, palm oil, and castor oil. Mixtures of vegetable oils can be binary, ternary, quaternary, or higher mixtures.

In one embodiment, the vegetable oil is present in the composition at a concentration of about 0.1% (w/w) to about 10% (w/w). In a further embodiment, the vegetable oil is present in the composition at a concentration of about 1% (w/w) to about 8% (w/w). In a further embodiment, the vegetable oil is present in the composition at a concentration of about 1% (w/w) to about 6% (w/w). In a further embodiment, the vegetable oil is present in the composition at a concentration of about 1% (w/w) to about 4% (w/w). In one embodiment, the vegetable oil is present in the composition at a concentration of about 0.1% (w/w), about 0.5% (w/w) about 1.0% (w/w), about 1.25% (w/w), about 1.5% (w/w), about 1.75% (w/w), or about 2.0% (w/w).

In one embodiment, the topical composition provided herein comprises a vegetable oil and at least one cellulose derivative. For example, in one embodiment, the topical composition comprises hydroxypropyl cellulose and a vegetable oil, or hydroxyethyl cellulose and a vegetable oil, or a combination of hydroxypropyl cellulose, hydroxyethyl cellulose, and a vegetable oil. In one embodiment, the cellulose derivative and the vegetable oil (e.g., palm, oil or corn oil), are each present at the same concentration (w/w). In a further embodiment, the cellulose derivative and the vegetable oil are each present in the composition at about 1% (w/w) to about 5% (w/w). In even a further embodiment, the cellulose derivative is a combination of hydroxypropyl cellulose and hydroxyethyl cellulose, and each is present in the composition at about 1.25% (w/w). In one embodiment, the composition comprises a vegetable oil and two cellulose derivatives. In a further embodiment, the two cellulose derivatives are hydroxypropyl cellulose and hydroxyethyl cellulose, and the total concentration of cellulose derivatives in the composition is about 1.25% (w/w). Cellulose derivatives include, for example, hydroxyethyl cellulose, hydroxypropyl cellulose, methylcellulose, ethylcellulose, hydroxypropyl methyl cellulose, or cellulose acetate.

In some embodiments, the topical composition provided herein comprises one or more accelerants. In a further embodiment, the accelerant is an organic acid, a chelator, an anti-bacterial agent, an anti-fungal agent, an anti-viral agent, or a combination thereof. In a further embodiment, the accelerant is a chelator. In even a further embodiment, the accelerant is EDTA.

The accelerant, in one embodiment, is EDTA. In a further embodiment, the GML composition provided herein comprises EDTA at a concentration of about 0.00005 M, about 0.0005 M, about 0.005 or about 0.05 M. In another embodiment, a chelator is present in the composition at a concentration of about 0.00005 M to about 0.05 M. about 0.0005 M to about 0.005 M, or about 0.005 to about 0.05 M.

In one embodiment, the topical composition comprises both a vegetable oil and an accelerant, for example, palm oil and EDTA. In another embodiment, the accelerant is an organic acid and is present in the formulation with a vegetable oil. In one embodiment, the topical composition provided herein comprises an accelerant and a non-aqueous gel, for example a gel comprising a cellulose derivative. In another embodiment, the topical composition comprises GML or a derivative thereof, a vegetable oil, a non-aqueous gel (e.g., a gel comprising one or more cellulose derivatives) and an accelerant.

In one embodiment, the composition contains at least one pharmaceutically acceptable excipient. Pharmaceutically acceptable excipients are well known to those skilled in the art and may include buffers (e.g., phosphate buffer and citrate buffer), amino acids, alcohols, proteins such as serum albumin, parabens (e.g., methylparaben), or mannitol.

In one embodiment, the pH of the composition is from about 3.5 to about 7.0. In a further embodiment, the pH of the composition is from about 4.0 to about 6.0. In a still further embodiment, the pH of the composition is from about 4.0 to about 4.5.

In one embodiment, the composition provided herein comprises GML or a derivative thereof and a pharmaceutically acceptable topical carrier. In one embodiment, the pharmaceutically acceptable topical carrier is a mix of hydrocarbons such as, for example, paraffin wax or petroleum jelly. Petroleum jelly is any water-insoluble, hydrophobic, semi-solid mixture of hydrocarbons. The pharmaceutically acceptable topical carrier can be added to any of the formulations described herein.

In another embodiment, the composition comprises an aqueous solvent. Compositions comprising an aqueous solvent may or may not include a pharmaceutically acceptable topical carrier. In one embodiment, the aqueous solvent is present, and is water, saline, growth medium (e.g., microbial culture medium or cell culture medium), or a combination thereof. In a further embodiment, both an aqueous solvent and pharmaceutically acceptable topical carrier are present in the topical composition. In even a further embodiment, the topical composition comprises at least one cellulose derivative.

In one embodiment, the composition comprises bacterial culture media such as Todd Hewitt media as the aqueous solvent. In one embodiment, the aqueous solvent is present at a concentration of about 1% (w/w) to about 25% (w/w). In a further embodiment, the aqueous solvent is about 2% (w/w) to 5% (w/w) of the composition.

In one embodiment, the composition is a liquid solution. In another embodiment, the composition is a gel. In another embodiment, the composition is a solid, semi-solid, foam, wax, cream, or lotion.

In one embodiment, the composition comprises one of the formulations provided in Table 1.

The vegetable oil, in one embodiment, is palm, olive or corn vegetable oil. It should be noted that Table 1 is merely exemplary of the composition components and concentrations that can be used with the disclosed application.

TABLE 1
Exemplary GML Formulations

| | | Concentration | |
|---|---|---|---|
| Formulation | Components | (if applicable) | |
| 1 | GML | 0.001%-10% w/v | |
| | | (10 μg/mL-100 mg/mL) | |
| | Cellulose derivative | | |
| | 0.1% (w/w) to 5.0% (w/w) | | |
| | Vegetable Oil | Up to 100% | w/v |
| 2 | GML | 0.001%-10% w/v | |
| | | (10 μg/mL-100 mg/mL) | |
| | Non-aqueous Gel | 10%-25% | w/v |
| | Polyethylene glycol | | |
| | 400 (20% to | | |
| | 35%(w/w)) | | |
| | Propylene glycol | | |
| | (65% to 80% (w/w)) | | |
| | Cellulose derivative | | |
| | (0.5% to 5.0% (w/w)) | | |
| | Water or saline | Up to 100% | w/v |
| 3 | GML | 0.001%-10% w/v | |
| | | (10 μg/mL-100 mg/mL) | |
| | Non-aqueous Gel | 10%-25% | w/v |
| | Polyethylene glycol | | |
| | 400 (20% to | | |
| | 35%(w/w)) | | |
| | Propylene glycol | | |
| | (65% to 80% (w/w)) | | |
| | Cellulose derivative | | |
| | (0.5% to 5.0% (w/w)) | | |
| | Water or saline | 1% to 25% | w/v |
| | Vegetable oil | Up to 100% | w/v |
| 4 | GML | 0.001%-10% w/v | |
| | | (10 μg/mL-100 mg/mL) | |
| | Non-aqueous Gel | 10%-25% | w/v |
| | Polyethylene glycol | | |
| | 400 (20% to | | |
| | 35%(w/w)) | | |
| | Propylene glycol | | |
| | (65% to 80% (w/w)) | | |
| | Cellulose derivative | | |
| | (0.5% to 5.0% (w/w)) | | |
| | Vaseline | up to 100% | w/v |
| 5 | GML | 5% | w/v |
| | Non-aqueous Gel | | |
| | Polyethylene glycol | | |
| | 400 (25%(w/w)) | | |
| | Propylene glycol | 10% | w/v |
| | (73.55% (w/w)) | | |
| | Cellulose derivative | | |
| | (1.25% (w/w)) | | |
| | Water | 85% | w/v |
| 6 | GML | 100 | μg/mL |
| | Vegetable oil | Up to 100% | w/v |

In one embodiment is provided a method of treating a microbial infection in a subject in need thereof. The microbial infection, in one embodiment, is a bacterial, viral, fungal, or protozoan infection, or a combination thereof. In one embodiment, the microbial infection is caused by bacteria. In another embodiment, is provided a method of treating a viral infection in a subject in need thereof. In one embodiment, the microbial infection is caused by fungus. In one embodiment, the microbial infection is caused by protozoa.

In some embodiments, the method of treating a microbial infection is the microbiological infection that is sexually transmitted. In some embodiments, the sexually transmitted disease is gonorrhea or Zika or both.

In some embodiments, the bacterial infection can be caused by the genus Neisseria. Exemplary species include Neisseria gonorrheae. In some embodiments, the compositions are used to treat infections caused by Neisseria gonorrhoeae, and includes, without limitation, cervical, urethral, rectal and pharangeal infections, multidrug resistant infections, and both uncomplicated and complicated infections.

In one embodiment, the microbial infection is caused by bacteria Zika virus replicates in the mosquito's midgut epithelial cells and then its salivary gland cells. After 5-10 days, Zika virus can be found in the mosquito's saliva, which can then infect humans. If the mosquito's saliva is inoculated into human skin, the virus can infect epidermal keratinocytes, skin fibroblasts in the skin and the Langerhans cells. The pathogenesis of the virus is hypothesized to continue with a spread to lymph nodes and the bloodstream. Flaviviruses generally replicate in the cytoplasm, but Zika antigens have been found in infected cell nuclei. The Zika virus has also been shown to be sexually transmitted in humans.

In some embodiments, the method of treating a microbial infection is the microbiological infection that is sexually transmitted. In some embodiments, the sexually transmitted disease is trichomoniasis.

Disclosed are compositions, methods for treatment of viral infection (e.g., caused by the viruses described herein). The compositions can contain glycerol monolaurate (G-ML) that may be administered, e.g., by topical administration. The compositions and methods, in one embodiment, are used for treating viral infections topically, for example, by facilitating delivery of effective amounts of GML or a derivative thereof to a skin or mucosal surface of a subject, e.g., a human. In certain embodiments, the viral infection may be caused by a virus that is a member of one or more of the following groups: single stranded RNA viruses, flaviviridae viruses (e.g., a Zika). In certain embodiments, the viral infection is caused by the Zika virus.

The GML topical compositions described herein are less irritating than currently approved antimicrobial compositions, therefore may result in a more favorable patient compliance rate, as compared to other antimicrobial compositions presently used in the art.

In one embodiment, the method comprises administering to the subject a topical composition comprising GML or a derivative thereof, as described herein. In one embodiment, the method comprises topically administering to the subject an effective amount of a composition comprising GML or a derivative thereof (e.g., a compound of one of Formulae I-VI), a vegetable oil, and a pharmaceutically acceptable topical carrier. In another embodiment, the method comprises topically administering an effective amount of a composition comprising GML, a non-aqueous gel, and a pharmaceutically acceptable topical carrier. In yet another embodiment, the method comprises administering to the subject one of the compositions provided in Table 1.

In one embodiment, the method of treating a microbial infection comprises applying an effective amount of one or more of the GML compositions described herein to at least one skin or mucosal surface of a subject. In one embodiment, the compositions are administered topically to the teeth and gum, skin, nasal, or vaginal areas.

In some embodiments, the composition is applied to or impregnated in a wipe, sponge, swab, or other material, and then applied to the skin or mucosal surface of the subject using the respective material. In some embodiments, the material is attached to a holder, for example a stick, wire, rod, or applicator. In further embodiments, the material attached to a holder is attached at one or both ends thereof. In some embodiments, the wipe, sponge, swab, or other material is pre-loaded or packaged together with the composition.

GML inhibits microbial infection through one or more of several mechanisms that include, but are not limited to, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

Bacteria use two-component signal transduction systems to respond and adapt to environmental changes as well as produce virulence factors. GML interferes with bacterial signal transduction, either directly or indirectly, through interaction with bacterial plasma membranes. In one embodiment, GML's bactericidal effect is mediated at least in part by interactions at the bacterial plasma membrane. In a further embodiment, GML can be detected in association with the bacterial plasma membrane, but cannot be detected in association with the cytoplasm.

In one embodiment, direct GML-mediated interruption of bacterial membranes includes interference with the localization of signaling proteins within the membrane, or interference with ligand binding to signaling proteins. In one embodiment, GML has an indirect effect on a two-component signal transduction system and the effect is selected from modifications to membrane structure that interfere with the ability of transmembrane proteins to perform signaling functions; dissipation of the bacterial plasma membrane potential; and alterations of pH gradients across the membranes.

In a further embodiment, the patient has undergone long-term antibiotic therapy prior to the topical application of the composition.

In some embodiments, the subject has a bacterial infection. Bacterial infections that are treatable with the topical compositions provided herein include, infections caused by genus Neisseriae (e.g. Neisseria gonorrheae).

Methods of identifying and diagnosing a bacterial, viral, or fungal infection are generally known by those skilled in the art. To assess whether the formulations disclosed herein are useful to treat an infection, methods known to those of ordinary skill in the art may be employed. For example, a gonorrhoeae infection prior to, and after treatment, may be assessed by microscopic examination of vaginal cells.

Methods of identifying and diagnosing a viral infection are also generally known by those skilled in the art. To assess whether the disclosed formulations are useful to treat an infection, methods known to those of ordinary skill in the art may be employed. For example, a virus infection prior to, and after treatment, may be assessed by virus isolation or RNA detection.

In some embodiments, the methods of the invention comprise administering a second or additional active agent, along with GML or a derivative of GML. The additional active agent may be present in the compositions described herein, or may be administered separately. In one embodiment, the one or more additional active agents prior to, or after, the topical GML composition is administered. For example, the two active agents may be topically administered serially, or administered serially by different routes of administration.

In one embodiment, the additional active agent(s) is administered before, during, or after administration of the composition. In another embodiment, the additional active agent(s) is administered by the same route as the composition or by a different route. For example, the additional active agent(s), in one embodiment, is administered by one of the following routes of administration: topical, intranasal, intradermal, intravenous, intramuscular, oral, vaginal, rectal, otic, ophthalmic, subcutaneous. The dose of additional active agents depends on, for example, the nature of the infection or illness; the site of administration; subject weight, age, sex, and surface area; concomitant medications; and medical judgment.

Additional active agents include, for example, antibiotics, anti-viral agents, and anti-fungal agents. Antibiotics include, without limitation, aminoglycosides, carbacephems, cephalosporins, glycopeptides, lincosamides, lipopetides, macrolides, monobactams, nitrofurans, penicillins, polypetides, quinolones, sulfuramides, and tetracyclines.

Anti-fungal agents include, without limitation, those of the azole class, polyene class, or echinocanins class, nucleoside analogues, allylamines, griseofulvin, tolnaftate, or selenium compounds.

Anti-viral agents include, for example and without limitation, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, and zanamivir.

In one embodiment, the present invention is directed to a composition comprising glycerol monolaurate (GML) or a derivative thereof, and a vegetable oil. In one embodiment, the vegetable oil is palm, olive, corn, canola, coconut, soybean, or wheat, or a combination thereof. In a further embodiment, the vegetable oil is present in the composition at about 10% to about 99%, about 20% to about 90%, about 30% to about 80%, or about 40% to about 70%. In one embodiment, the composition comprising GML or a derivative thereof and a vegetable oil further comprises a pharmaceutically acceptable topical carrier, for example, petroleum jelly. In one embodiment, GML or a derivative thereof is present in the composition at a concentration from about 10 μg/mL to about 100 mg/mL, from about 50 μg/mL to about 50 mg/mL, from about 100 μg/mL to about 10 mg/mL, or from about 500 μg/mL to about 5 mg/mL. In another embodiment, the composition comprising GML or a derivative thereof and a vegetable oil further comprises a cellulose derivative, for example either hydroxypropyl cellulose or hydroxyethyl cellulose, or a combination thereof. In a further embodiment, the cellulose derivative is present in the composition up to 1.25% w/w.

In another embodiment, the present invention is directed to a composition comprising GML or a derivative thereof, and a non-aqueous gel. In one embodiment, the composition comprising GML or a derivative thereof and a non-aqueous gel has a pH of about 4.0 to about 4.5. In one embodiment, the non-aqueous gel comprises polyethylene glycol, hydroxypropyl cellulose, hydroxyethyl cellulose, or a combination thereof. In a further embodiment, the polyethylene glycol is present at about 25% w/w in the composition. In one embodiment, hydroxypropyl cellulose and hydroxyethyl cellulose are both present in the composition, each at a concentration of about 1.25% w/w.

In one embodiment, the GML composition comprising a non-aqueous gel comprises polyethylene glycol with a molecular weight range of about 300 to about 4000. In a further embodiment, the polyethylene glycol has a molecular weight of about 400 or about 1000.

In one embodiment, the GML composition comprising a non-aqueous gel further comprises a topical carrier, e.g., petroleum jelly. In a further embodiment, the composition comprises a vegetable oil.

In one embodiment, the compositions described herein comprise GML or a derivative thereof at a concentration of about 0.001% (w/v) to about 10% (w/v) of the total composition. In a further embodiment, GML or a derivative thereof is present at about 0.005% (w/v) to about 5% (w/v) of the composition. In a further embodiment, GML or a derivative thereof is present at about 0.01 to about 1%. In a still further embodiment, GML or a derivative thereof is present at about 0.1% (w/v) to about 0.5% (w/v) of the composition.

In one embodiment, GML or a derivative thereof is present in the composition at a concentration of about 10 μg/mL to about 100 mg/mL. In a further embodiment, GML or a derivative thereof comprises about 50 μg/mL to about 50 mg/mL of the composition. In a further embodiment, GML or a derivative thereof comprises about 100 μg/mL to about 10 mg/mL. In a still further embodiment, GML or a derivative thereof comprises about 500 μg/mL to about 5 mg/mL.

In one embodiment, the GML composition provided herein comprises propylene glycol at a concentration of about 65% (w/w) to about 80% (w/w). In another embodiment, polyethylene glycol is present in the composition at a concentration of about 20% (w/w) to about 35% (w/w). In one embodiment, both propylene glycol and polyethylene glycol are present in the topical composition.

In one embodiment, the composition includes a cellulose derivative. In a further embodiment, the composition comprises hydroxypropyl cellulose or hydroxyethyl cellulose. In a yet further embodiment, the cellulose is present at a concentration of about 0.1% (w/w) to about 5.0% (w/w).

In one embodiment, the GML composition comprises an aqueous solvent. In a further embodiment, the aqueous solvent is water, saline, media, or a combination thereof.

In one embodiment, the pharmaceutically acceptable topical carrier is petroleum jelly.

In one embodiment, the pH of the GML composition provided herein is from about 4.0 to about 5.5.

In some embodiments, the composition provided herein comprises one or more accelerants. Accelerants may be an organic acid including, without limitation, lactic acid, ascorbic acid, citric acid, formic acid, benzoic acid, and oxalic acid. The accelerant, in another embodiment, is a chelator, and in one embodiment, is selected from ethylenediaminetetraacetic acid (EDTA), dimercaprol, dimercaptosuccinic acid (DMSA), 2,3-dimercapto-1-propanesulfonic acid (DMPS), alpha lipoic acid (ALA), or combinations thereof. In another embodiment, the accelerant is selected from an antibiotic agent, anti-fungal agent, anti-viral agent, or combination thereof.

In a further embodiment, the accelerant is an organic acid, a chelator, an anti-bacterial agent, an anti-fungal agent, an anti-viral agent, or a combination thereof. In a further embodiment, the accelerant is a chelator. In even a further embodiment, the accelerant is EDTA.

In another embodiment, the GML composition provided herein has anti-microbial, anti-viral, and/or anti-inflammatory activity.

Accordingly, in one embodiment, are methods for treating a microbial infection in a subject in need thereof. In one embodiment, the method comprises topically administering to the subject in need thereof, an effective amount of a GML composition provided herein.

In one embodiment are methods of treatment or prophylaxis of a sexually transmitted disease in a subject, wherein the sexually transmitted disease is caused a bacteria or virus, the method comprising administering to the subject a composition comprising an effective amount of glycerol monolaurate and derivatives thereof and one or more pharmaceutically acceptable excipients.

In one embodiment, the composition comprises GML or a derivative thereof, a vegetable oil, and a pharmaceutically acceptable topical carrier. In another embodiment, the composition comprises GML or a derivative thereof, a non-aqueous gel, and a pharmaceutically acceptable topical carrier. In a further embodiment, the composition comprises GML or a derivative thereof, a vegetable oil, a non-aqueous gel, and a pharmaceutically acceptable topical carrier.

In one embodiment, the compositions disclosed herein are applied topically with the use of a sponge, wipe, or swab.

In another embodiment, the method of the invention involves administering a second active agent selected from the group consisting of anti-fungal agents, anti-viral agents, and antibiotics.

In some embodiments, the composition includes one or more additional or second agent, which includes the genus of bacteria Lactobacillus. In other embodiments, the composition may include bacteria from the genus Lactobacillus. Lactobacilli are gram positive rods that are a part of the microbial flora of the human gut, mouth, and vagina. Vaginal lactobacilli are thought to play an important role in resistance to infection via production of lactic acid and acidification of the vagina or by production of other antimicrobial products, such as hydrogen peroxide H2O2. It has been demonstrated that women with predominant vaginal Lactobacillus flora have a 50% lower frequency of gonorrhea, chlamydial infections, trichomoniasis and bacterial vaginosis. The presence of H2O2-producing Lactobacilli in the vagina have been linked to a decreased frequency of bacterial vaginosis, symptomatic yeast vaginitis and sexually transmitted pathogens including Neisseria gonorrhea, Chlamydia trachomatis, and Trichomonas vaginalis. In vitro studies have demonstrated that H2 O2-producing Lactobacilli have potent bactericidal and viricidal properties against vaginal pathogens and even against human immunodeficiency virus (HIV).

In one embodiment, the composition may include bacteria from the genus Lactobacillus. Exemplary species from this genus include Lactobacillus crispatus and Lactobacillus jensenii. In some embodiments, the Lactobacillus strain has all of the identifying characteristics of Lactobacillus crispatus and includes Lactobacillus crispatus CTV-05 and Lactobacillus crispatus SJ-3C strain deposited under ATCC number PTA-10138.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1-23. (canceled)

24. A method of treatment or prophylaxis of a sexually transmitted disease in a subject, wherein the sexually transmitted disease is caused a by a bacterium, a fungus, or a protozoan, the method comprising:
administering to the subject a composition comprising an effective amount of glycerol monolaurate and derivatives thereof and one or more pharmaceutically acceptable excipients.

25. The method of claim 24, wherein the glycerol monolaurate is administered to the subject at a dosage of 0.0001-100 mg/kg.

26. The method of claim 24 further comprising an accelerant selected from an organic acid, a chelator, an antibiotic agent, an anti-fungal agent, an anti-viral agent, an anti-protozoan agent, or a combination thereof.

27. The method of claim 26, wherein the one or more accelerants comprise ethylenediaminetetraacetic acid (EDTA) or ascorbic acid.

28. The method of claim 24 further comprises a pharmaceutically acceptable topical carrier.

29. The method of claim 28, wherein the pharmaceutically acceptable topical carrier comprises propylene glycol, polyethylene glycol, or petroleum jelly.

30. The method of claim 24 further comprising administering one or more additional or second agents before, simultaneous with, or after the administration of the glycerol monolaurate.

31. The method of claim 30, wherein the one or more additional or second agent comprises antibiotics, or a genus of bacteria Lactobacillus.

32. The method of claim 24, wherein the administering the composition is by topical application.

33. The method of claim 24, wherein the composition further comprises vegetable oil.

34. The method of claim 33, wherein the vegetable oil is palm oil, olive oil, corn oil, canola oil, coconut oil, soybean oil, wheat germ oil, or a combination thereof.

35. The method of claim 24, wherein the sexually transmitted disease is gonorrhea or Zika.

36. The method of claims 24, wherein the sexually transmitted disease is caused by the genus Neisseria.

37. The method of claim 24, wherein the sexually transmitted disease is caused by Neisseria gonorrheae bacterial microorganism.

38. The method of 24, wherein the sexually transmitted disease is caused by the genus Flavivirus.

39. The method of claim 24, wherein the composition comprises:
about 73.55 w/w % propylene glycol;
about 25 w/w % polyethylene glycol 400;
about 1.25 w/w % hydroxyethyl cellulose or hydroxypropyl cellulose; and
about 1-25 w/w % saline and/or water.

40. The method of claim 24, wherein the composition comprises an accelerant and:
about 73.55 w/w % propylene glycol;
about 25 w/w % polyethylene glycol 400;
about 1.25 w/w % hydroxyethyl cellulose or hydroxypropyl cellulose; and
about 1-25 w/w % saline and/or water.

41. The method of claim 24, wherein the subject is human.
